(19) United States
(12) Patent Application Publication
(10) Pub. No.: US 2012/0111332 A1
(43) Pub. Date: May 10, 2012

(54) BREATHING APPARATUS

(76) Inventors: Michael H. Gusky, Weston, FL (US); Jeffrey Walter Bentzler, Playa Del Rey, CA (US); Dennis Lynn Schroeder, Los Angeles, CA (US); Stuart Allan Karten, Venice, CA (US)

(21) Appl. No.: 13/289,178

(22) Filed: Nov. 4, 2011

Related U.S. Application Data
(60) Provisional application No. 61/410,134, filed on Nov. 4, 2010.
(60) Provisional application No. 61/423,195, filed on Dec. 15, 2010.

(51) Int. Cl. A61M 16/06

(52) U.S. Cl. 128/205.25

(57) ABSTRACT

A breathing apparatus includes an air delivery assembly and a nasal interface. The air delivery assembly includes at least one passage couple to a supply of air. The nasal interface includes a resilient pad having a first side configured to releasably couple with the air delivery assembly. A second side of the resilient pad is configured to engage at least a portion of a users nose. The nasal interface further includes a first air passage and a second air passage. The first air passage and the second air passage are configured to provide an air pathway between the air delivery assembly and a respective first nasal passage and second nasal passage of the user.

### CROSS REFERENCE TO RELATED APPLICATIONS

This application is related to and claims the benefit of priority of U.S. Provisional Patent Application No. 61/410,134 and U.S. Provisional Patent Application No. 61/423,195, which are hereby incorporated by reference.

### TECHNICAL FIELD

The present disclosure generally relates to a breathing apparatus, and more particularly relates to nasal interface for delivering air to a user.

### BACKGROUND OF THE DISCLOSURE

Various products have been developed for the treatment of snoring and of sleep apnea. One common approach is directed at maintaining positive airway pressure of a user in an attempt to prevent the closing of the user's airways. One general variety of positive airway pressure devices is the continuous positive airway pressure (CPAP) system, which seeks to maintain a constant pressure in the user's upper airways. In providing a pressurized air to a user, in order to maintain a positive pressure in the user's upper airways, it is necessary to provide a nasal interface which engaged the user's nose or nasal passages, so as to minimize uncontrolled air leakage between the breathing apparatus and the user's upper airways.

Many typical nasal interface configurations include features that may engage the nasal passages of the user to provide a relatively air-tight connection between the breathing apparatus and the user's nasal airways. As users may have greatly varying facial geometries and nasal passage sizes, typically a variety of sizes of nasal interfaces must be provided, to provide an acceptable fit for various users. However, even with an acceptably fitting nasal interface, movements of the user during sleep may cause relative movement of the breathing apparatus and the user's face. Such relative movements may often result in air leakage and diminished performance of the breathing apparatus, noise, etc.

### SUMMARY OF THE DISCLOSURE

According to a first implementation a breathing apparatus includes an air delivery assembly and a nasal interface. The air delivery assembly includes at least one passage couple to a supply of air. The nasal interface includes a resilient pad. The resilient pad includes a first side configured to releasably couple with the air delivery assembly. The resilient pad also includes a second side configured to engage at least a portion of a nose of a user. The nasal interface further includes a first air passage and a second air passage, the first air passage and the second air passage configured to provide an air pathway between the air delivery assembly an a respective first nasal passage and second nasal passage of the user.

One or more of the following features may be included. The resilient pad may include a gel material. The resilient pad may include an elastomeric material. One or more of the air delivery assembly and the nasal interface may include an adhesive layer for releasably coupling the nasal interface with the air delivery assembly. The air delivery assembly and the resilient pad may include cooperating interlocking features for releasably coupling the nasal interface with the air delivery assembly.

The air delivery assembly may include two air delivery assembly openings. Each of the two air delivery assembly openings may be configured to at least partially correspond to a respective one of first air passage and the second air passage. The resilient pad may include a substantially flat second side. The second side of the resilient pad may be configured to at least partially conform to at least a portion of a nose of the user. The first air passage and the second air passage may each include a protrusion on the second side of the resilient pad configured to be at least partially received within each respective nasal passage of the user.

The breathing apparatus may include a first nasal prong and a second nasal prong. The first nasal prong and the second nasal prong may have a respective first end configured to be at least partially received within a respective one of the first air passage and the second air passage, and may have a respective second end configured to be at least partially received within a respective one of the first nasal passage and the second nasal passage of the user. The first nasal prong and the second nasal prong may include a resilient material. The second end of the first nasal prong and the second nasal prong may include an enlarged, distally tapering profile configured to engage the respective first nasal passage and second nasal passage of the user.

According to another implementation, a breathing apparatus includes an air delivery assembly and a nasal interface. The air delivery assembly includes at least one passage coupled to a supply of air. The nasal interface includes a resilient pad configured to releasably couple with the air delivery assembly. The resilient pad has a substantially flat interface surface configured to at least partially conform to at least a portion of a nose of a user. The nasal interface further defines a first air passage and a second air passage configured to provide a respective first air pathway between the air delivery assembly and a first nasal passage of the user and a second air pathway between the air delivery assembly and a second nasal passage of the user.

One or more of the following features may be included. One or more of the air delivery assembly and the nasal interface may include an adhesive for releasably coupling the resilient pad with the air delivery assembly. The resilient pad may include a gel material. The resilient pad may include an elastomeric material. The air delivery assembly may include at least one opening configured to be at least partially sealed by the nasal interface. The first and second air passage may be configured to fluidly couple with the at least one opening of the air delivery assembly.

According to yet another implementation, a breathing apparatus includes an air delivery assembly and a nasal interface. The air delivery assembly includes at least one passage coupled to a supply of air. The nasal interface includes a resilient pad configured to releasably couple with the air delivery assembly. A first nasal prong and a second nasal prong are each configured to be at least partially received in the resilient pad. The first nasal prong and the second nasal prong are also configured to provide a first air pathway and a second air pathway between the at least one passage of the air delivery assembly and a respective first nasal passage of a user and a second nasal passage of a user.

One or more of the following features may be included. The resilient pad may include a gel material. The resilient pad may include an elastomeric material. The first nasal prong and the second nasal prong may include a resilient material. The first nasal prong and the second nasal prong may include an enlarged portion configured to be at least partially received in the respective first nasal passage of the user and second nasal passage of the user. The first nasal prong and the second nasal prong may be at least partially independently movable relative to one another.

The details of one or more implementations are set forth in the accompanying drawings and the description below. Other features and advantages will become apparent from the description, the drawings, and the claims.

### BRIEF DESCRIPTION OF THE DRAWINGS

FIG. 1 depicts an exploded view of an embodiment of a breathing apparatus.

FIG. 2 schematically shows a side view of the breathing apparatus of FIG. 1.

FIG. 3 depicts another embodiment of a breathing apparatus.

FIG. 4 shows an exploded view of the breathing apparatus of FIG. 3.

FIGS. 5 through 7 depict various views of yet another embodiment of a breathing apparatus.

FIG. 8 is a cross-sectional view of still a further embodiment of a breathing apparatus.

FIG. 9 depicts an exploded view of a nasal interface of the breathing apparatus of FIG. 8.

FIG. 10 depicts a schematic view of an embodiment of an air supply tube.

FIG. 11 depicts a schematic view of an embodiment of pre-shaped air supply tubes.

FIG. 12 depicts a schematic side view of an embodiment of an example of pre-shaped air supply tubes.

FIG. 13 depicts a schematic view of an embodiment of an example nasal interface with an adhesive surface.

FIG. 14A-FIG. 14D depict schematic views of an embodiment of an example nasal interface.

FIG. 15 depicts a schematic view of an alternative embodiment of a breathing apparatus.

FIG. 16 depicts a schematic view of an alternative embodiment of a breathing apparatus.

FIG. 17 depicts a schematic view of an embodiment of an embodiment of an example of pre-shaped air supply tubes.

FIG. 18 depicts a schematic side view of an embodiment of an embodiment of an example of pre-shaped air supply tubes.

FIG. 19 depicts a schematic perspective front view of an embodiment of the FIG. 18 embodiment of an example of pre-shaped air supply tubes.

FIG. 20 depicts a schematic perspective rear view of an embodiment of the FIG. 18 embodiment of an example of pre-shaped air supply tubes.

FIG. 21A depicts a schematic view of an embodiment of an example of a pre-shaped air supply tube.

FIG. 21B depicts a schematic view of the FIG. 21A embodiment of an example of a pre-shaped air supply tube as it can be worn by a user.

FIG. 22 depicts a schematic view of an embodiment of an example of pre-shaped air supply tubes including user controls.

FIG. 23 depicts a schematic view of an embodiment of an example of pre-shaped air supply tubes integrated into a headband.

FIG. 24 depicts a schematic perspective view of an embodiment of an example of pre-shaped air supply tubes.

FIG. 25 depicts a schematic perspective view of an embodiment of an example of pre-shaped air supply tubes.

FIG. 26 depicts a schematic perspective view of an embodiment of an example of pre-shaped air supply tubes.

FIG. 27 depicts a schematic perspective view of an embodiment of an example of pre-shaped air supply tubes including cheek pads.

FIG. 28 depicts a schematic perspective view of an embodiment of an example of pre-shaped air supply tubes.

FIG. 29 depicts a schematic perspective view of an embodiment of an example of pre-shaped air supply tubes including a headphone type member.

FIG. 30 depicts a schematic perspective view of an embodiment of an example of pre-shaped air supply tubes.

### DETAILED DESCRIPTION OF EXEMPLARY EMBODIMENTS

In general, a breathing apparatus may include an air delivery assembly and a nasal interface. The air delivery assembly may include at least one passage coupled to a supply of air, such as a CPAP apparatus, or similar breathing air supply apparatus. A nasal interface may include a resilient pad having a first side that may be configured to be releasably coupled with the air delivery assembly. The resilient pad may include a second side configured to engage at least a portion of a nose of a user. The nasal interface may further include a first air passage and a second air passage, in which the first air passage and the second air passage may be configured to provide an air pathway between the air delivery assembly (e.g., which may be provided with a supply of air from a CPAP or similar apparatus) and a respective first nasal passage and second nasal passage of a user.

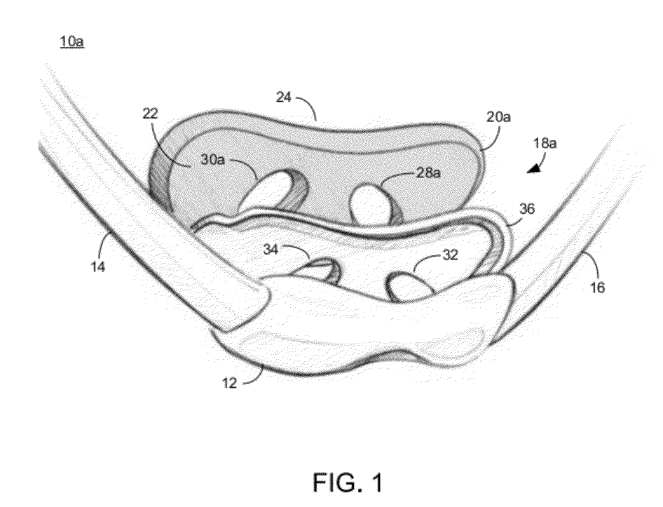

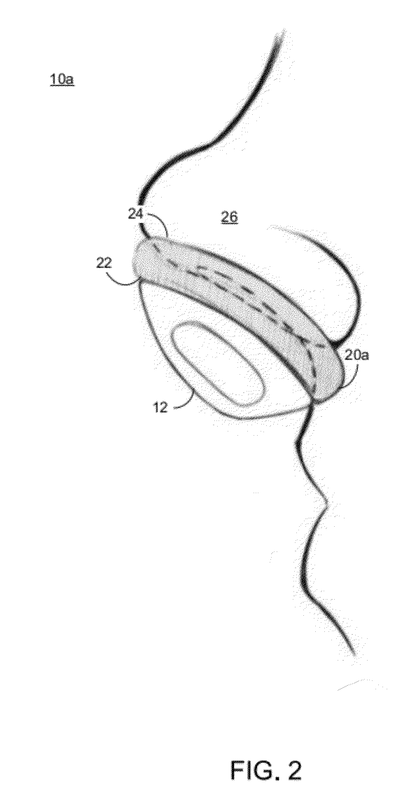

For example, and referring to FIGS. 1 and 2, breathing apparatus 10a may include air delivery assembly 12. Air delivery assembly 12 may include at least one passage (e.g., air supply tubes 14, 16) that may be coupled to a supply of air. As discussed above, various suitable supplies of air may include various continuous positive airway pressure systems, as well as various other suitable breathing air supplies. Breathing apparatus 10a may further include a nasal interface (nasal interface 18a, generally). Nasal interface 18a may include resilient pad 20a. One side (e.g., first side 22) of resilient pad 20a may be configured to releasably couple with air delivery assembly 12. A generally opposed side of resilient pad 20a (e.g., second side 24) may be configured to engage at least a portion of nose 26 of a user. Nasal interface 18a may further include first air passage 28a and second air passage 30a. As shown, first air passage 28a and second air passage 30a may include an opening formed through resilient pad 20. First air passage 28a and second air passage 30a may be configured to provide an air pathway between air delivery assembly 12 and a respective first nasal passage and second nasal passage (not shown) of the user.

While not shown, breathing apparatus 10a may include an associated headgear. The headgear may be coupled to breathing apparatus 10a and may hold breathing apparatus 10a in position relative to a user's face. As such, the headgear may maintain breathing apparatus 10a such that first air passage 28a and second air passage 30a may generally align with the nasal passages of the user. Resilient pad 20a may allow breathing apparatus 10a to sealingly engage at least a portion of nose 26 of the user, to thereby provide the air pathway between delivery assembly 12 (and thereby providing an air pathway between the user and the supply of air). In this regard, resilient pad may include a material that may at least partially conform to at least a portion of nose 26 of the user, to thereby aid breathing apparatus 10a in sealingly engaging at least a portion of nose 26 of the user. In various embodiments resilient pad 20a may include a gel material (e.g., a silicone gel, or other similar gel material), or an elastomeric material (e.g., a medical grade silicone elastomer), which may at least partially conform to at least a portion of nose 26 of the user. Resilient pad 20a may include gel materials and/or elastomeric materials of various density, compliance, and durometer according to a general desired degree of compliance and pressure applied by the headgear. Such characteristics may generally be based upon design criteria and user preference, such as individual comfort and the like.

In the particular embodiment shown in FIGS. 1 and 2, second side 24 of resilient pad 20a may be substantially flat. As used herein, flat is intended to mean a generally even surface without substantial prominences or depressions. As such, substantially flat second side 24 may include a curved, or compound curved, geometry (e.g., to better correspond with a general geometry of nose 26), but without any substantial prominences or depressions from a nominal surface of second side 24. According to such a configuration, sealing engagement between nasal interface 18a and nose 26 of the user may be based upon, at least in part, at least partial conformance between at least a portion of resilient pad 20a (e.g., at least a portion adjacent first air passage 28a and second air passage 30a) and at least a portion of nose 26 of the user.

Consistent with the foregoing embodiment, breathing apparatus 10 may provide a breathing apparatus that may be relatively easily adapted to suit a variety of users. For example, in addition to varying the nature of resilient pad 20a to suit various users and various facial geometries, the size, location, orientation, etc., of first air passage 28a and second air passage 30a may be varied to suit various users and various facial geometries. For example, an array of resilient pads may be available having different air passage configurations. As resilient pad 20a may be configured to be releasably coupled with air delivery assembly 12, a resilient pad having a first air passage configuration may be replaced with a resilient pad having a different air passage configuration to suit a given user. Additionally/alternatively, because resilient pad 20a may be configured to releasably couple with air delivery assembly, resilient pad 20a may be regularly replaced for comfort, hygiene, etc., without the need to replace the entirety of breathing apparatus 10a. In this regard, resilient pad 20a may be a consumable and/or disposable component.

In one embodiment, one or more of resilient pad 20a and air delivery assembly 12 may include an adhesive layer (not shown), which may releasably couple resilient pad 20a with air delivery assembly 12. For example, resilient pad 20a may include a pressure sensitive adhesive disposed on at least a portion of first side 22. Accordingly, with the pressure sensitive adhesive on first side 22 exposed (e.g., via the removal of a protective release layer, not shown), resilient pad 20a may be positioned relative to air delivery assembly 12 and pressed onto air delivery assembly 12 to releasably couple resilient pad 20a with air delivery assembly 12. In a similar manner, resilient pad 20a may be relatively easily removed from air delivery assembly 12, e.g., by peeling resilient pad 20a from air delivery assembly 12.

Air delivery assembly 12 may include at least one opening configured to be at least partially sealed by nasal interface 18a. First and second air passages 28a, 30a may be configured to fluidly couple with the at least one opening of air delivery assembly 12. For example, and as shown in FIG. 1, air delivery assembly 12 may include two air delivery assembly openings (e.g., first air delivery assembly opening 32, and second air delivery assembly opening 34), which may, for example, be disposed in top plate 36. First air delivery assembly opening 32 and second air delivery assembly opening 34 may generally be configured to at least partially correspond to first air passage 28a and second air passage 30a, thereby providing an air pathway there-between. In various embodiments, first air delivery assembly opening 32 and second air delivery assembly opening 34 may be larger or smaller than a given first air passage 28a and second air passage 30a. As such, first air delivery assembly opening 32 and second air delivery assembly opening 34 may accommodate variously sized and positioned first air passages and second air passages (e.g., which may be sized and shaped to accommodate various facial geometries, and the like).

While air delivery assembly 12 is shown including two openings (namely first air delivery assembly opening 32 and second air delivery assembly opening 34) it should be appreciated that air delivery assembly 12 may include only a single opening which may generally correspond to, and provide a fluid pathway with, both first air passage 28a and second air passage 30a. Further, in addition to providing first air delivery assembly opening 32 and second air delivery assembly 34, top plate 36 of air delivery assembly 12 may provide an adequate surface area for releasably coupling resilient pad 20a with air delivery assembly 12 (e.g., via an adhesive or the like). As shown, top plate 36 may include a member which may be couple to (e.g., snap fit, adhesively bonded, welded or the like) to air delivery assembly 12. Various additional/alternative configurations may equally be utilized (e.g., a single unitary air delivery assembly, or the like).

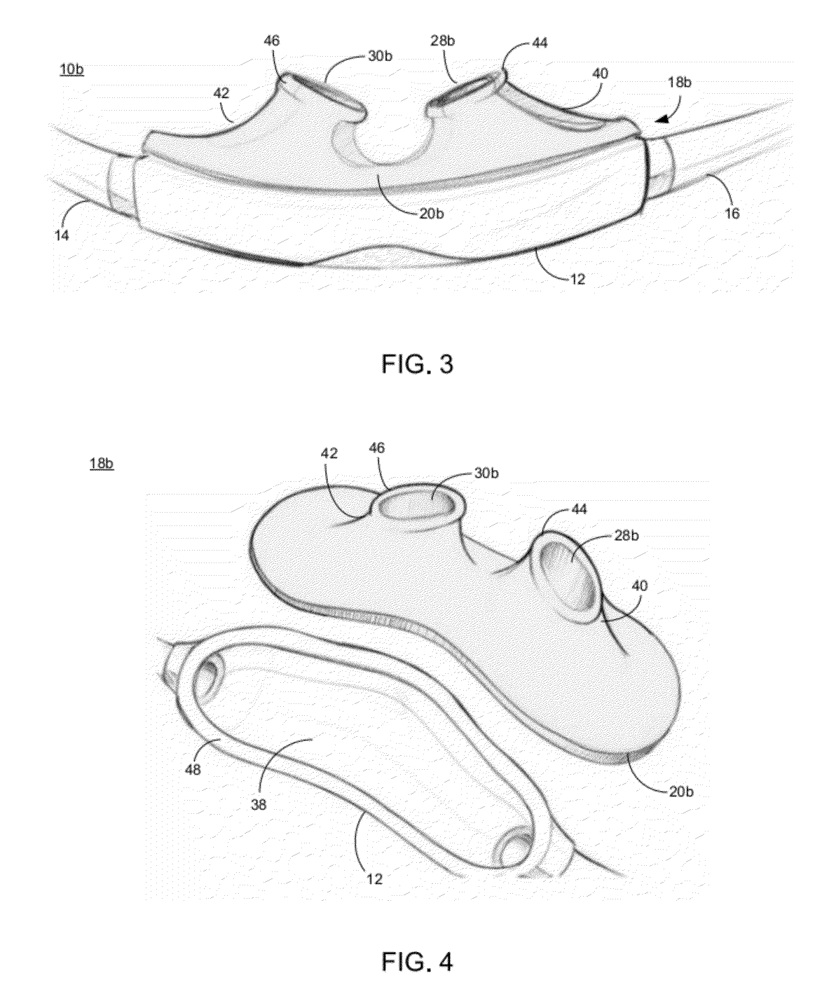

Referring to FIGS. 3 and 4, in a related embodiment breathing apparatus 10b similarly includes air delivery assembly 12 and a resilient pad (e.g., resilient pad 20b). As with the previous embodiment, air delivery assembly 12 may include at least one passage (e.g., air supply tubes 14, 16) that may be coupled to a supply of air. Air delivery assembly 12 may include at least one opening (e.g., opening 38) configured to be at least partially sealed by nasal interface 18a (e.g., including resilient pad 20b). Additionally, resilient pad 20b may be configured to be releasably coupled with air delivery assembly 12.

As shown in FIGS. 3 and 4, rather than having a substantially flat second side, resilient pad 20b may include first air passage 28b and second air passage 30b, which may each include a protrusion (e.g., protrusions 40, 42) on second side 24 of resilient pad 20b. Protrusions 40, 42 may be configured to be at least partially received within each respective nasal passage of user. As shown, protrusions 40, 42 may be integrally formed with resilient pad 20b (e.g., and may be formed of the same material as resilient pad 20b). As such, protrusions 40, 42 may be configured to at least partially conform to at least a portion of an interior of a respective nasal passage of the user. As such, protrusions 40, 42 may at least partially sealingly engage the nasal passages of the user. To this end, in some embodiments (and as shown in FIGS. 3 and 4) protrusions 40, 42 may each include a distal bead (e.g., distal beads 44, 46). Distal beads 44, 46 may at least partially resist deformation. Accordingly, when at least partially inserted into a respective nasal passage of the user, distal beads 44, 46 may at least partially resist deformation, which may increase the engagement between protrusions 40, 42 and the interior of the nasal passages of the user.

As with the previous embodiment, breathing apparatus 10b may utilize an adhesive to releasably couple resilient pad 20b with air delivery assembly 12. Additionally/alternatively, air delivery assembly 12 and resilient pad 20b may include cooperating interlocking features for releasably coupling the nasal interface 18b with air delivery assembly 20b. For example, resilient pad 20b may include a groove (not shown) that may cooperate with bead 48 of air delivery assembly 12 to releasably couple resilient pad 20b and air delivery assembly 12. In a similar manner, resilient pad 20b may be sized to be at least partially received within opening 38 of air delivery assembly 12 (e.g., to provide an at least partial interference fit), thereby releasably coupling resilient pad 20b with air delivery assembly 12. Various additional/alternative embodiments will also be appreciated. While not shown in the preceding embodiment, any of the various embodiments of the breathing apparatus may utilize adhesive, interlocking features, etc., for releasably coupling the resilient pad with the air delivery assembly.

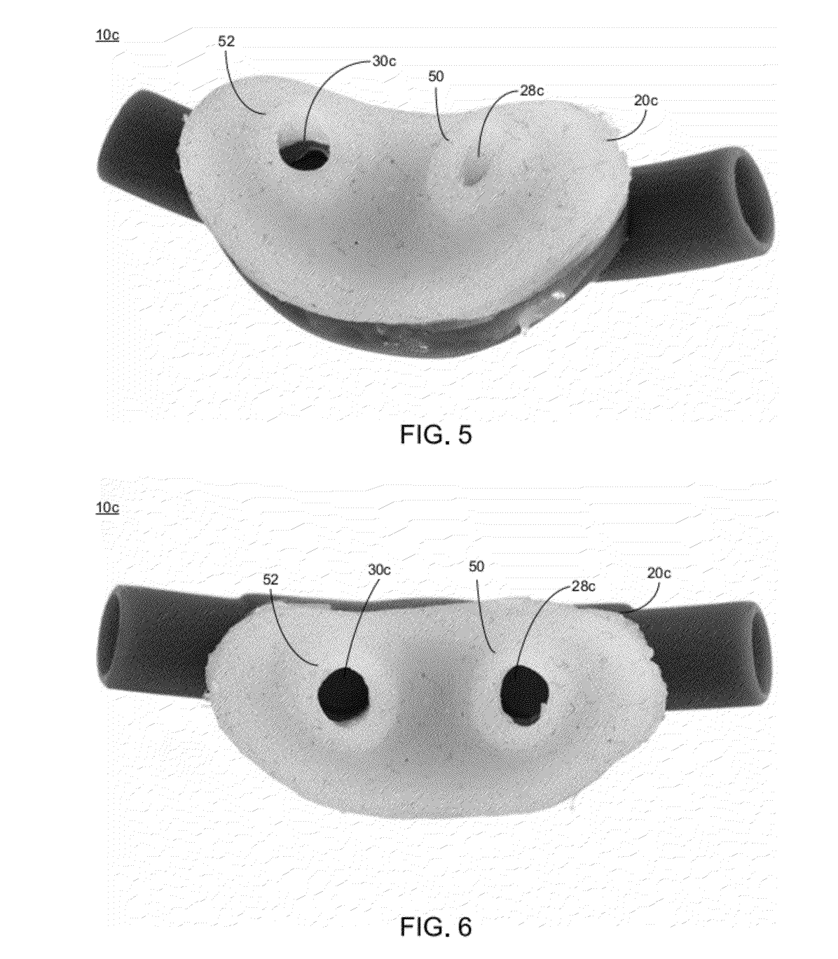

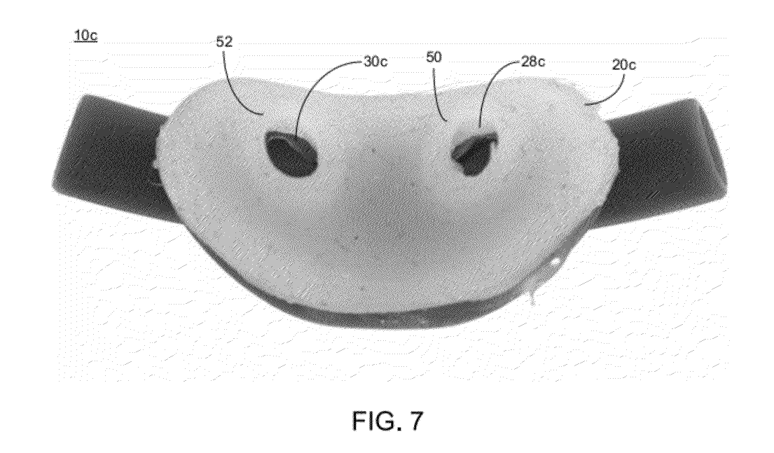

Referring also to FIGS. 5 through 7, in a related embodiment, breathing apparatus 10c may include resilient pad 20c. Similar to the embodiment shown in FIGS. 3 and 4, resilient pad 20c may include first air passage 28c and second air passage 30c. First air passage 28c and second air passage 30c may include bumps 50, 52. Bumps 50, 52 may be integrally formed with resilient pad 20c (and may, therefore, be made of the same material as resilient pad 20c). Bumps 50, 52 may be positioned to be at least partially received in a respective nasal passage of the user. As such, bumps 50, 52 may at least partially locate breathing apparatus 10c relative to the nose of the user. Additionally, bumps 50, 52, as well as the surrounding portions of second side 24 of resilient pad 20c, may conform to at least a portion of the nose of the user. As such, bumps 50, 52 may assist in sealingly engaging breathing apparatus to the upper airways of the user.

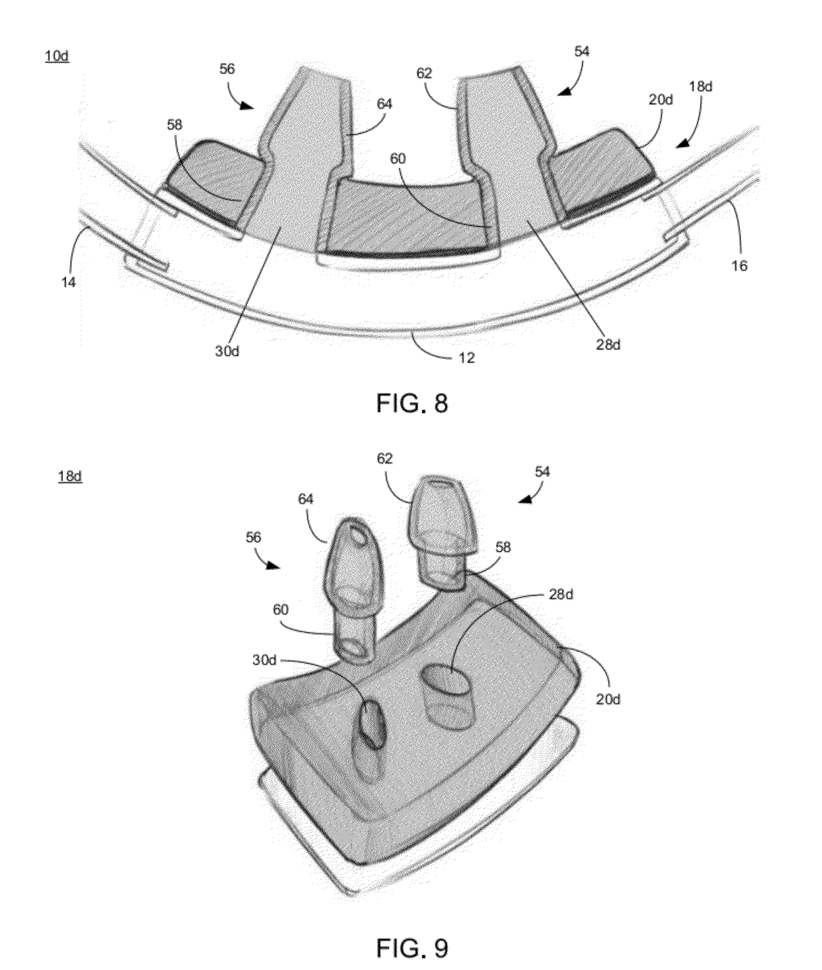

Referring next to FIGS. 8 and 9, in another embodiment breathing apparatus 10d may include first nasal prong 54 and second nasal prong 56. First nasal prong 54 and second nasal prong 56 may include a respective first end (e.g., first ends 58, 60, respectively) that may be configured to be at least partially received within a respective one of first air passage 28d and second air passage 30d of resilient pad 20d. As first ends 58, 60 or first nasal prong 54 and second nasal prong 56 may be at least partially received within a respective one of first air passage 28 and second air passage 30d of resilient pad 20d, first nasal prong 54 and second nasal prong 56 may be at least partially independently moveable relative to one another due to, at least in part, the resilient nature of resilient pad 20d. Accordingly, first nasal prong 54 and second nasal prong 56 may be capable of at least partial independent articulation. The at least partial independent articulation of first nasal prong 54 and second nasal prong 56 may allow breathing apparatus 10d to accommodate different facial geometries (e.g., including different nasal passage spacing, etc.), as well as accommodate at least some degree of movement between breathing apparatus 10d and the user without losing the sealing engagement between breathing apparatus 10d and the nasal passages of the user.

First nasal prong 54 and second nasal prong 56 may each have a second end (e.g., second ends 62, 64, respectively) that may be configured to be at least partially received within a respective one of the first nasal passage and the second nasal passage of the user, thereby providing an air passage between the upper airways of the user and air delivery assembly 12 (and the supply of air via air delivery assembly 12). As shown in FIGS. 8 and 9, second ends 62, 64 of first nasal prong 54 and second nasal prong 56 may include an enlarged, distally tapering profile. The enlarged, distally tapering profile may be configured to sealingly engage the respective first nasal pass and second nasal passage of the user. Further, at least the second ends 62, 64 of first nasal prong 54 and second nasal prong 56 may include a resilient material (e.g., a gel material, an elastomer, etc.). Accordingly, the enlarged distal ends of first nasal prong 54 and second nasal prong 56 may at least partially resiliently deform to conform to an interior geometry of the nasal passages of the user. Conforming to the interior geometry of the nasal passages of the user may, in some instances, improve the sealing engagement between first nasal prong 54 and second nasal prong 56 and the nasal passages of the user.

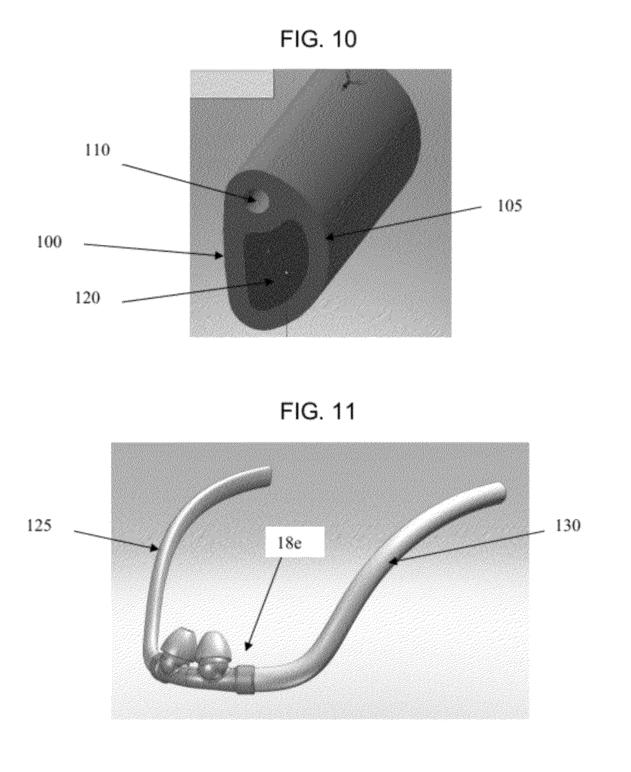

FIG. 10 depicts a schematic view of an embodiment of an air supply tube in accordance with aspects of the present invention. Commonly, techniques used to fix masks or nasal interfaces to deliver breathing gases are often complicated to adjust, cumbersome and make use of headgears composed of multiple straps. In common pressurized, closed systems the fixation of the mask or nasal interface is important to ensure a proper sealing of the nasal interface to the user's nose and thus eliminate or at least minimize leakage. Typically, breathing gases are delivered through circular shaped tubes of diameters larger than 10 mm for closed systems and about 4-7 mm for open systems. It is common that the fixation and the hoses/tubes carrying the breathing gas are separated, i.e. the hose does not function to fixing the nasal interface.

Exemplary embodiments of the present invention use alternative technologies to maximize user comfort by a combination of one or more of minimizing fixation apparatus, hose sizes, and the shape of the hoses. For example, in exemplary embodiments of the present invention the pre-shaped air supply tubes may be used to attached and fix the nasal interface to a user's nose. In other words, by using pre-shaped air supply tubes, there the use of additional straps or other means to hold the nasal interface in sealing contact with a user's nose are not needed. Of course, straps can be used if desired to augment the holding provided by embodiments of the present invention. Such straps may be attached at various locations such as, to the air supply tubes, or may be placed at other locations behind or above the head as discussed below.

Referring to FIG. 10, a cross-section, perspective view of an air supply tube includes a side 100. As shown in the exemplary embodiment illustrated in FIG. 10, the side 100 is substantially flat relative to another outside portion of the air supply tube, 105. This configuration allows the side 100 to contact a user and provide grater comfort that would be typically provided with a curved surface against the user. The shape of the air supply tube minimizes pressure spots on a user's skin. For example, the air supply tube shown in FIG. 10 has a “D” shape, with the side 100 corresponding to the straight part of the “D” and the other outside portion 105 corresponding to the curved portion of the “D.”

As shown in the exemplary embodiment of FIG. 10, the air supply tube can have more than one lumen, such as the lumens 110 and 120. In an example embodiment, the lumen 110 could be used to sense flow, pressure, humidity or other parameters directly at the nose and/or the face of the user. The lumen 120 can be used to supply air to the user.

In other embodiments, the air supply tube may be extruded with or without wires connected to various controls and/or sensors at the nose, the mouth, the face, or the head of a user. FIG. 11 depicts a schematic view of an embodiment of pre-shaped air supply tubes in accordance with aspects of the present invention. In the example of FIG. 11, the air supply tubes 125, 130 are pre-shaped to contour to a user's head, and connect to a nasal interface 18e. As seen in the FIG. 11 example embodiment, the inner part of the air supply tubes 125 and 130 are substantially flat relative to the outer, rounded outside portion of the air supply tubes 125, 130. The air supply tubes used in embodiments of the present invention may be coated with a material that makes them more skin friendly and less “sticky”, or gives them simply a better look.

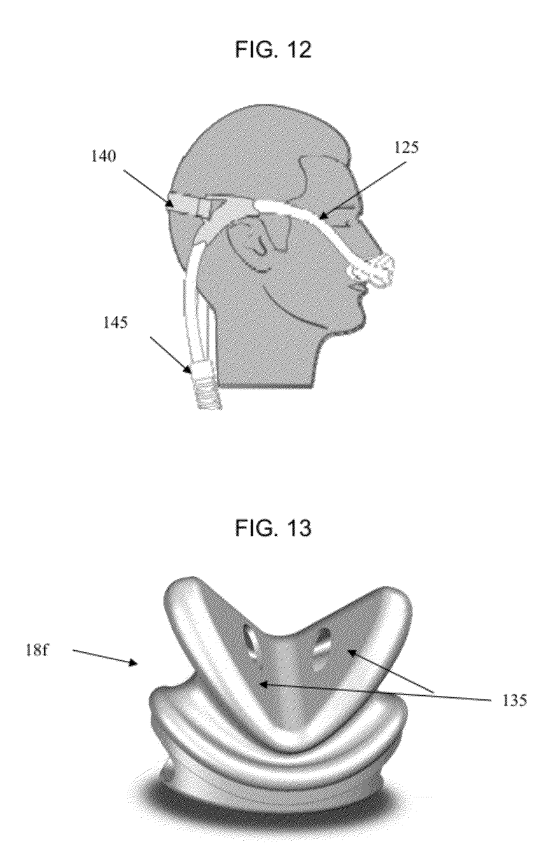

FIG. 12 depicts a schematic side view of a variation of the FIG. 11 embodiment of an example of pre-shaped air supply tubes in accordance with aspects of the present invention. In the FIG. 12 example, a supply tube 145 has a diameter greater than the air supply tube 125 running from the nose to behind the ear. This allows to optimization of comfort for the user by, for example making the part of the breathing apparatus in contact with the user's face as small as possible without generating too much flow resistance in a part of the breathing apparatus where the size of air supply tubing does not matter that much. The enlargement in diameter could be manufactured by, e.g., a thermo shape process where the larger part of the hose is generated by applying pressurized air to the heated tube.

The nasal interface depicted in the FIG. 12 embodiment can be the nasal interface shown in FIG. 13. Referring to the example embodiment of FIG. 12, the air supply tube 125 contours with the user's head, and connects with supply tube 145. The example embodiment of FIG. 12 also includes an optional auxiliary strap 140. As shown in the FIG. 12 example, the auxiliary strap 140 cooperates with the air supply tubes to hold the nasal interface in sealing engagement with the user's nose.

FIG. 13 depicts a schematic view of an embodiment of an example nasal interface 18f including an adhesive 135 formed on the surface of the nasal interface 18f. Because of the use of the structure of the nasal interface in accordance with embodiments of the present invention, alternate approaches to holding the nasal interface in place can be used. FIG. 13 and others discussed below are some examples. Obviously, the illustrated combinations of nasal interfaces and holding approaches (e.g., single or dual air supply tubes, with and without auxiliary straps) can be used in accordance with the present invention. The nasal interface 18f shown in FIG. 13 can be used without straps due to the structure of the nasal interface 18f and the use of the adhesive formed on the surface 135 of the nasal interface.

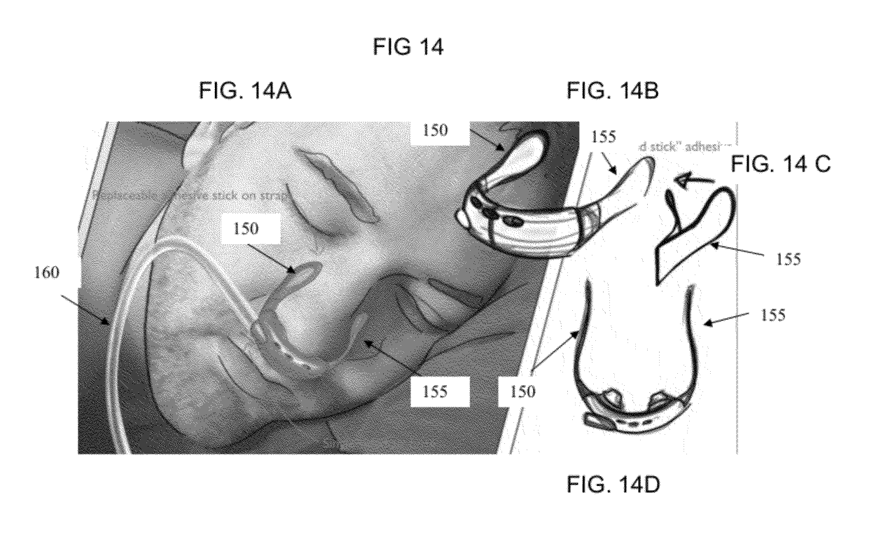

FIG. 14A-FIG. 14D depict schematic views of an embodiment of an example nasal interface in accordance with aspects of the present invention. FIG. 14A illustrates a nasal interface that includes adhesive strips 150 and 155. As shown in the example of FIG. 14C, the adhesive strips 150 and 155 can be a peel back type of adhesive strip. The air supply tube 160 shown in FIG. 14A can be a flexible tube of a pre-shaped tube. The adhesive strips 150 and 155 are configured, in the exemplary embodiments of FIG. 14A-FIG. 14D to attach to the side of a user's nose as illustrated in the example of FIG. 14A. FIG. 14D depicts a top view of the nasal interface shown in FIGS. 14A and 14B. As noted above, the adhesive strips 150 and 155 can be used with other nasal interfaces in accordance with the present invention, such as those illustrated in the other figures herein.

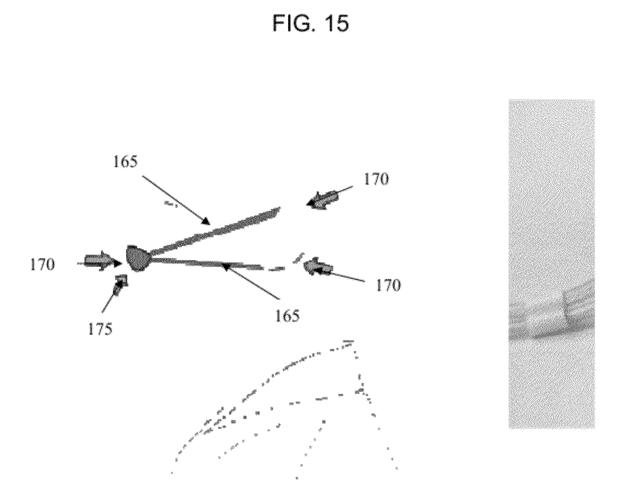

Because of the structure of the nasal interface in accordance with embodiments of the present invention, alternate approaches to holding the nasal interface in place can be used. FIG. 15 depicts a schematic view of an alternative embodiment of a breathing apparatus. FIG. 15 depicts a nasal interface being held in sealing engagement with the nose of a user via straps 165. FIG. 15 illustrate the direction of force by lines 170 that is provided by the behind the ear holding approach, and the direction of force 175 that the example arrangement provides.

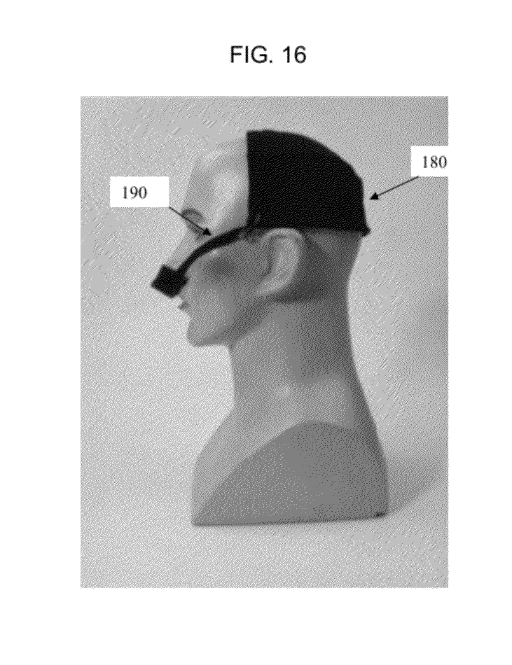

FIG. 16 illustrates an alternative holding arrangement in accordance with aspects of the present invention. In FIG. 16, a cap 180 in combination with straps 185 (only one shown in the FIG. 16 side view) holds a nasal interface in sealing engagement with the user's nose. This arrangement provides a customizable combination of a cap 180 and straps 185 for retention and comfort.

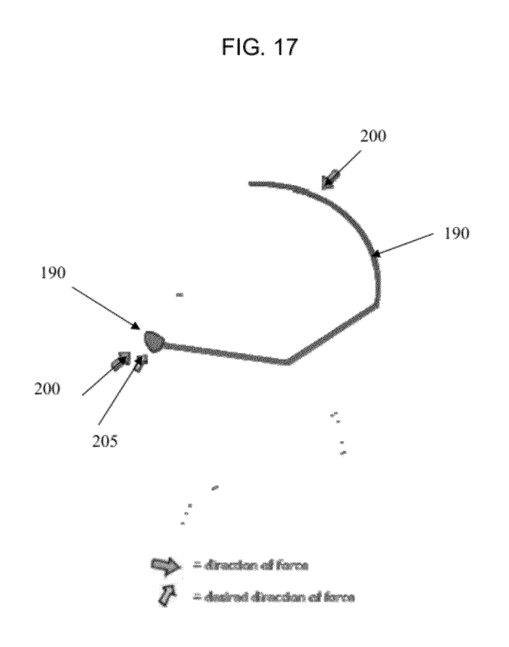

FIG. 17 schematically depicts a posable holding arrangement in accordance with aspects of the present invention. The exemplary FIG. 17 embodiment uses a combination of armature wire and tubing 190 to hold the nasal interface 195 in sealing engagement with the user's nose. As schematically illustrated in FIG. 17, the direction of the forces 200 holding the nasal interface in place align with the desired direction of force, 205.

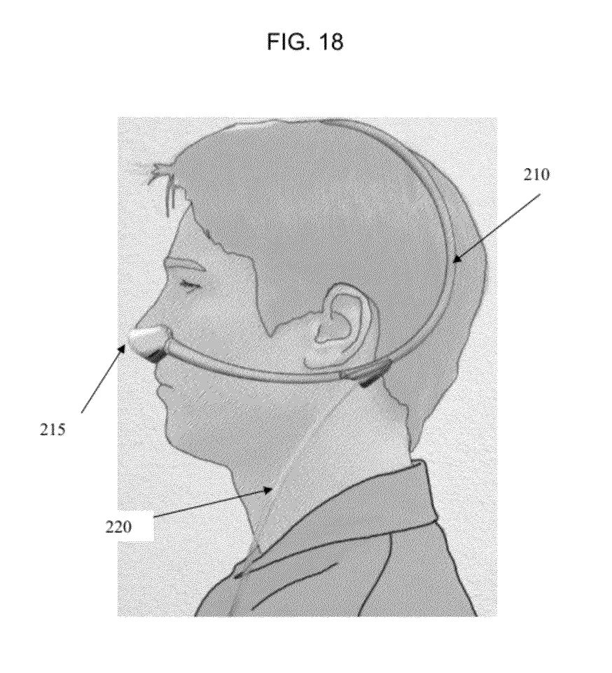

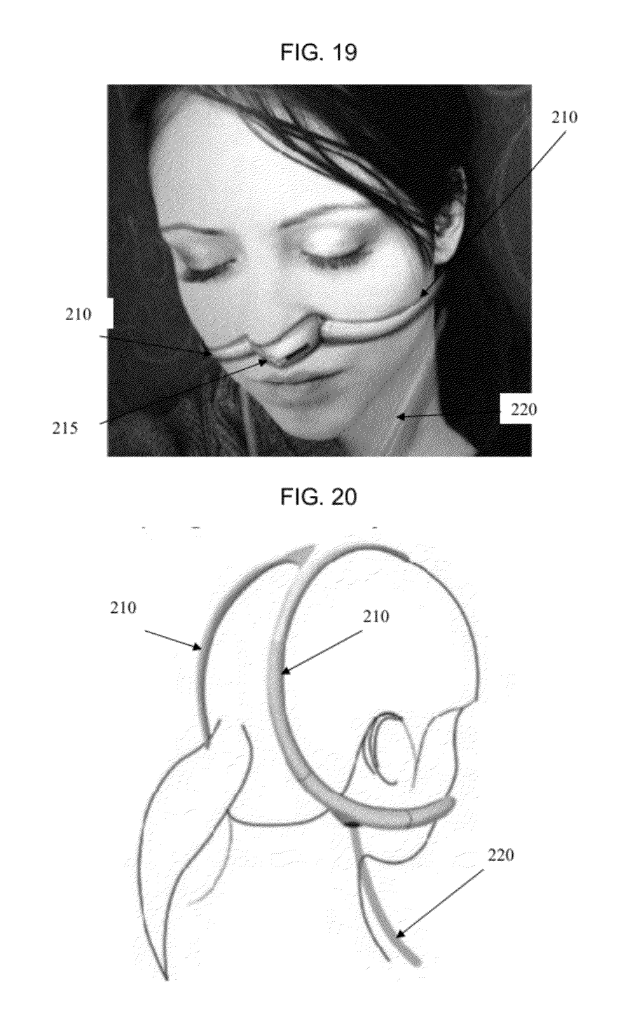

FIGS. 18, 19 and 20 are respectively a side view, front perspective view, and rear perspective view of an alternative arrangement of pre-shaped air supply tubes 210 in accordance with aspects of the present invention. Referring to FIG. 20, the exemplary air supply tubes 210 contour the user's head to hold the nasal interface 215 in sealing engagement with the user's nose as shown in FIG. 19. This exemplary arrangement can use, as shown in the figures, a flexible tube 220 to supply pressurized gas, such as air, to the user.

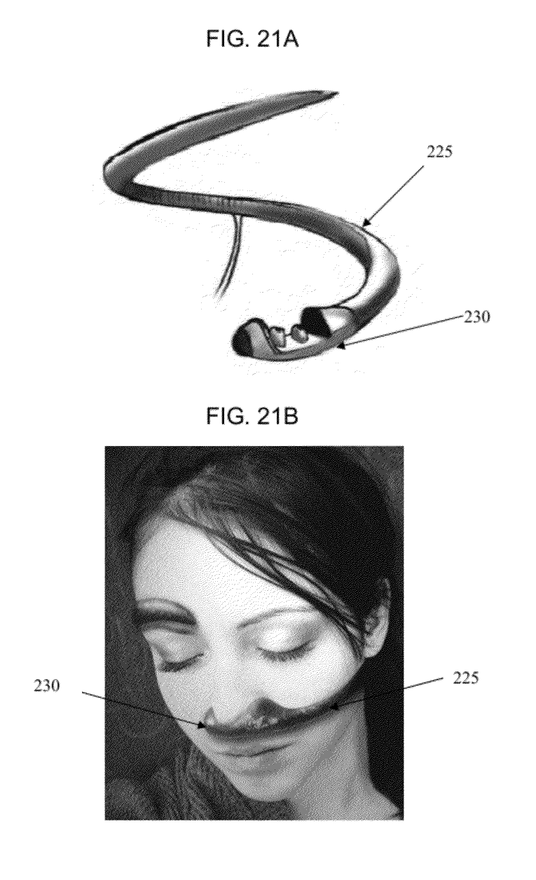

FIG. 21A depicts a schematic view of an embodiment of an example of an air supply tube 225 in accordance with aspects of the present invention. The shape of the air supply tube 225 shown in FIG. 21A contours the user's head as shown in FIG. 21B. The contour shape of the air supply tube 225 shown in the exemplary embodiment of FIG. 21A holds the nasal interface 230 in sealing engagement with the user's nose as shown in FIG. 21B.

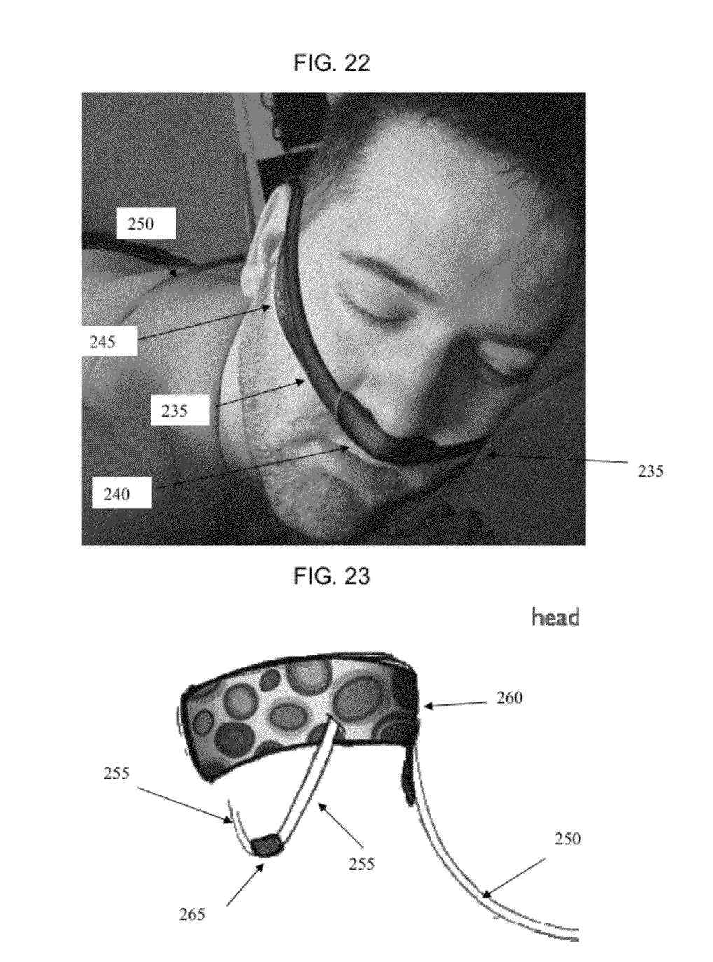

FIG. 22 schematically depicts a perspective view of an example of pre-shaped air supply tubes 235 holding a nasal interface 240 in sealing engagement with a user's nose. In the FIG. 22 embodiment, one air supply tube includes controls 245. Such controls can control, for example the air pressure within the nasal interface 240. The FIG. 22 embodiment uses flexible tubing 250 to supply pressurized gas, such as air, to the user.

FIG. 23 depicts a schematic view of an embodiment of an example of air supply tubes 255 integrated into a headband 260. As in the above embodiments, the air supply tubes 255 can, but need not be pre-shaped to aid in holding the interface 265 in sealing engagement with the user's nose. The FIG. 23 embodiment uses flexible tubing 250 to supply pressurized gas, such as air, to the user.

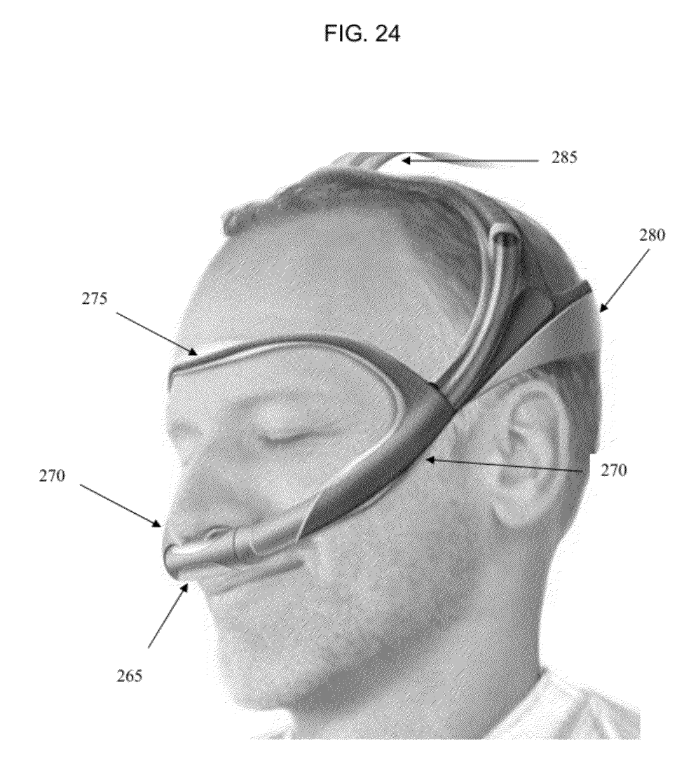

FIG. 24 depicts a schematic perspective view of another embodiment of an example of air supply tubes in accordance with aspects of the present invention. The FIG. 24 embodiment uses multi-durometer materials to provide a balance between structure and soft comfort for the user. In the FIG. 24 embodiment, air supply tubes 270 are pre-shaped and contour the user's head. The contour aids in holding the nasal interface 265 in sealing engagement with the user's nose. The exemplary FIG. 24 embodiment also uses an auxiliary cross member 275 that transverses the user's forehead. The auxiliary cross member 275 aids in positioning the nasal interface 265. The auxiliary member 280 traverses the back of the user's head and also aids in positioning the nasal interface. In the exemplary embodiment, the air supply tubes 270 together with the auxiliary cross member 275 and auxiliary member 280 cooperate to hold the nasal interface in sealing engagement with the user's nose. The FIG. 24 embodiment uses flexible tubing 285 to supply pressurized gas, such as air, to the user.

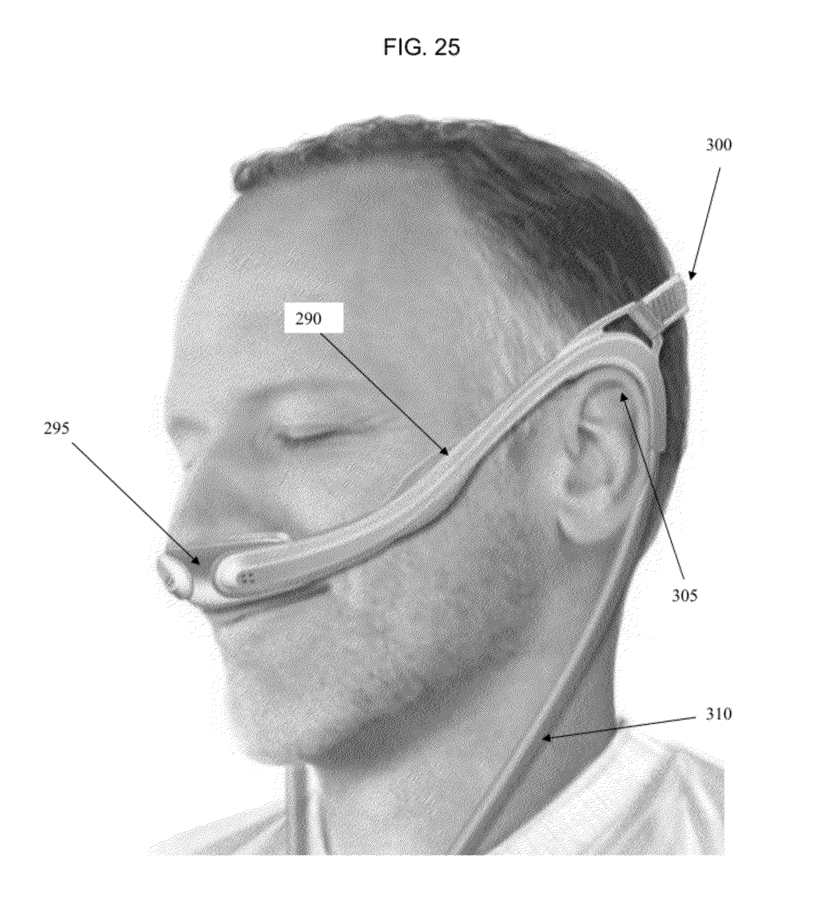

FIG. 25 depicts a schematic perspective view of an embodiment of an example of pre-shaped air supply tubes. In the exemplary embodiment shown in FIG. 25, pre-shaped air supply tubes 290 (only one is shown in FIG. 25) are contoured to the user's head. The air supply tubes 290 have a curved portion 305 that fits around a user's ear. The curved portion 305 together with a strap 300 hold the nasal interface 295 in sealing engagement with the user's nose as shown in FIG. 25. The FIG. 25 embodiment uses flexible tubing 310 to supply pressurized gas, such as air, to the user.

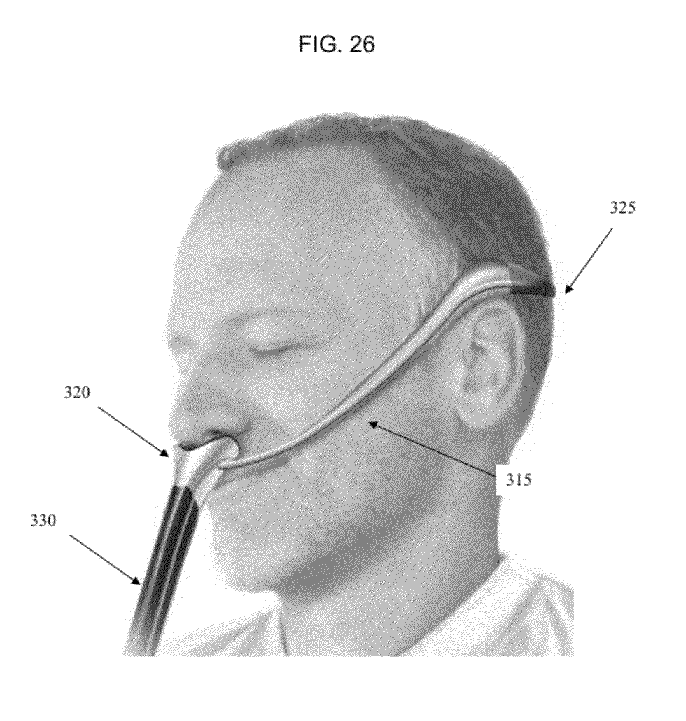

FIG. 26 depicts a schematic perspective view of an embodiment of an example of pre-shaped air supply tubes. The exemplary embodiment of FIG. 26 includes pre-shaped air supply tubes 315 (only one is shown in the perspective view of FIG. 26) that are contoured to the user's head. The exemplary embodiment of FIG. 26 also includes an auxiliary tube 330 that can supply pressurized gas, such as air, to the user. The auxiliary tube 330 can be used in stead of or in conjunction with air supply tube 315 to supply pressurized gas, such as air, to the user. When the auxiliary tube 330 is used as the source of pressurized gas, the air supply tubes function as a support member only to aid in positioning the nasal interface 320 in sealing engagement with the user's nose. The exemplary embodiment of FIG. 26 also includes a minimal back strap 325 to aid in positioning the nasal interface 320.

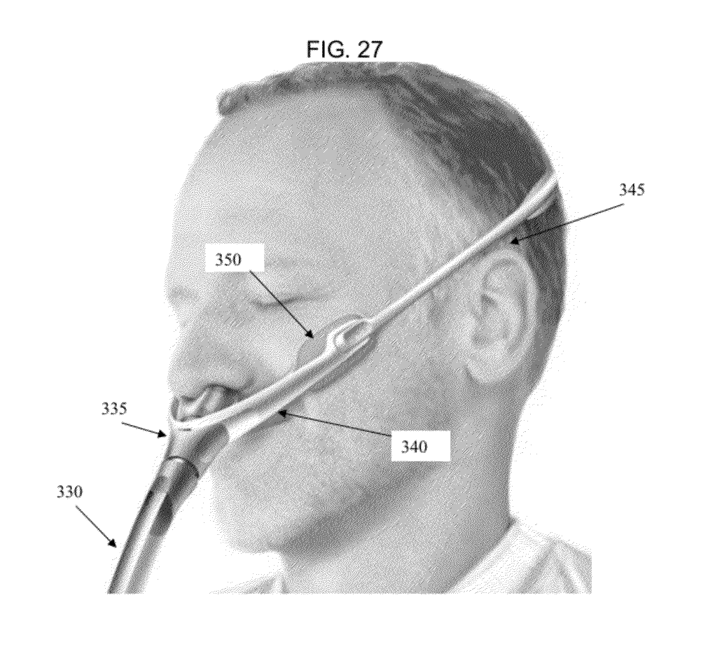

FIG. 27 depicts a schematic perspective view of an embodiment of an example of pre-shaped air supply tubes including cheek pads. In the exemplary embodiment shown in FIG. 27, the auxiliary tube 330 supplies pressurized gas, such as air, to the nasal interface 335 rather than the air supply tube 340. FIG. 27 illustrates only of the air supply tubes 340. In this exemplary embodiment, the air supply tubes 340 function as an extension of the nasal interface 335. The air supply tubes also provide a mechanism cooperate between the nasal interface and a strap 345 to hold the nasal interface in sealing engagement with the user's nose. For additional user comfort, the FIG. 27 embodiment also uses a cheek pad 350 (only one shown).

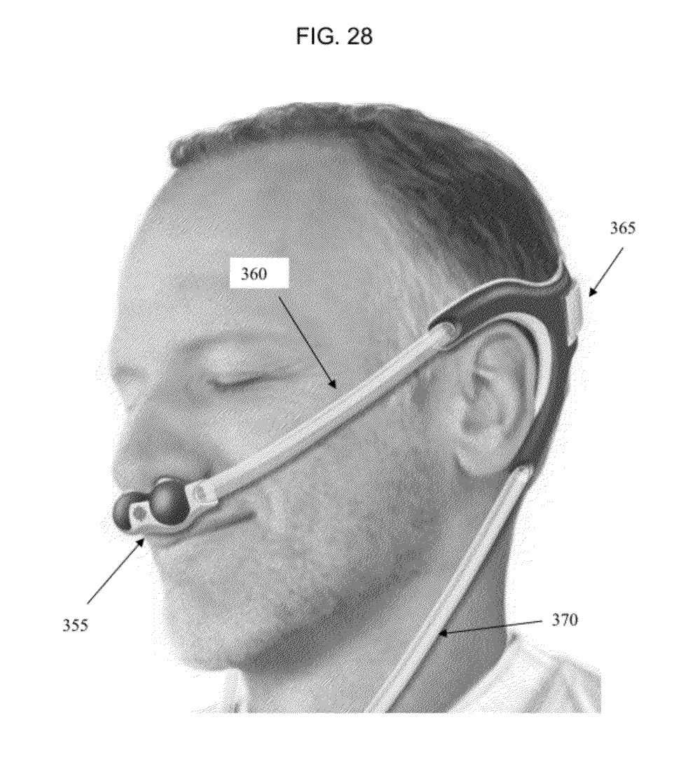

FIG. 28 depicts a schematic perspective view of an embodiment of an example of air supply tubes. The FIG. 28 embodiment is similar to that depicted in FIG. 25, but without the curved portion 305 of the FIG. 25 embodiment. FIG. 28 also uses a different nasal interface 355. This emphasizes that the various embodiments of the present invention illustrated and discussed herein are not limited to the specific nasal interface depicted and can be used with various other nasal interfaces without departing from the scope of the present invention.

Referring to FIG. 28 air supply tube 360 feeds through a strap 365. The perspective vies of FIG. 28 shows only one of the air supply tubes 360. The air supply tubes 360 cooperate with the strap 365 to hold the nasal interface in sealing engagement with the user's nose as shown in FIG. 28. The air supply tube can be pre-shaped or an extension of the flexible tube 370. The FIG. 28 embodiment uses flexible tubing 370 to supply pressurized gas, such as air, to the user.

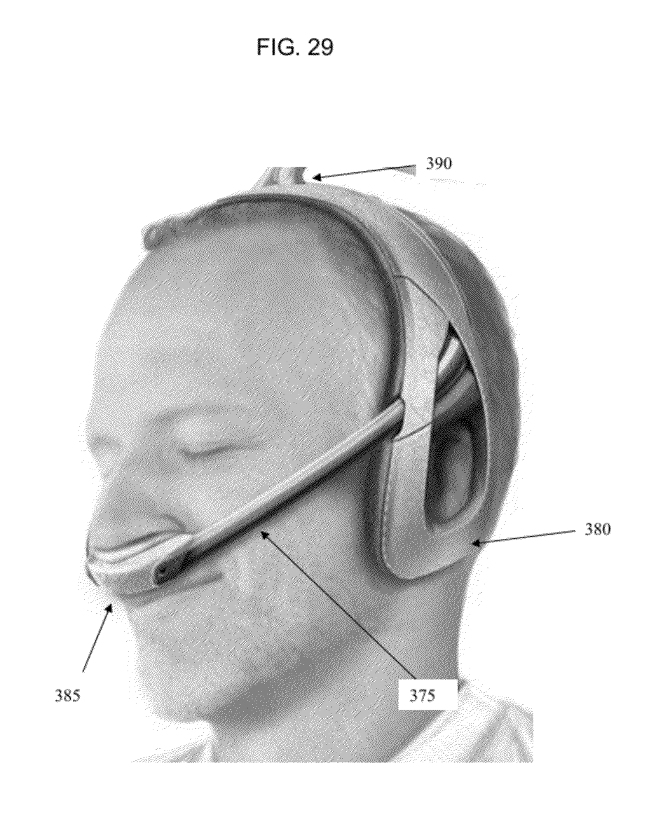

FIG. 29 depicts a schematic perspective view of an embodiment of an example of air supply tubes including a headphone type member 380. FIG. 29 illustrates a different nasal interface 385. This highlights that the various embodiments of the present invention illustrated and discussed herein are not limited to the specific nasal interface depicted and can be used with various other nasal interfaces without departing from the scope of the present invention.

Referring to FIG. 29, air supply tubes 375 are pre-shaped and (only one is shown in the perspective view of FIG. 29) contour the user's head. The air supply tubes 375 cooperate with the headphone member 380 to hold the nasal interface 385 in sealing engagement with the user's nose. The FIG. 29 embodiment uses flexible tubing 390 to supply pressurized gas, such as air, to the user.

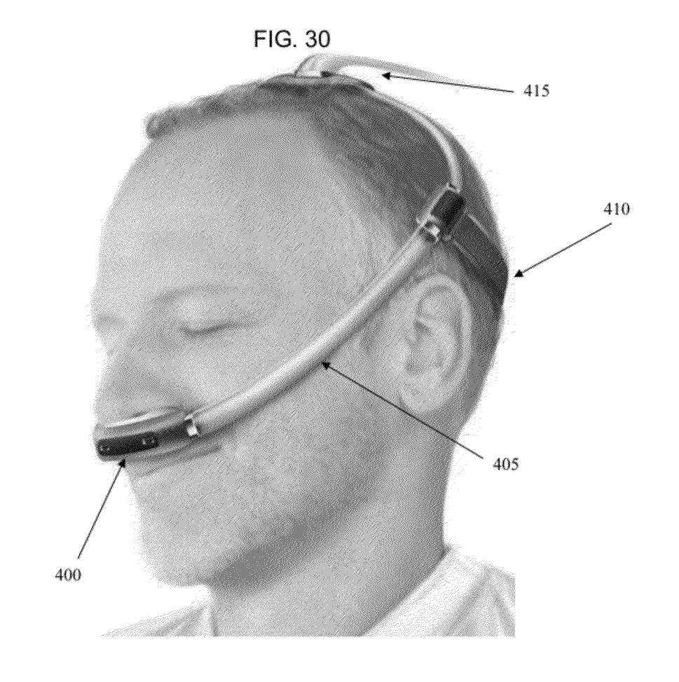

FIG. 30 depicts a schematic perspective view of an embodiment of an example of pre-shaped air supply tubes. FIG. 30 illustrates a different nasal interface 400. This highlights that the various embodiments of the present invention illustrated and discussed herein are not limited to the specific nasal interface depicted and can be used with various other nasal interfaces without departing from the scope of the present invention.

Referring to FIG. 30, air supply tubes 405 are pre-shaped and (only one is shown in the perspective view of FIG. 30) contour the user's head. The air supply tubes 405 cooperate with a strap 410 to hold the nasal interface 400 in sealing engagement with the user's nose. The FIG. 30 embodiment uses flexible tubing 415 to supply pressurized gas, such as air, to the user.

In the above embodiments, the exact straps, headbands, tubings are not needed for the particular embodiment and other variations of the disclosed structure may be used depending upon the particular design needed. These members may also include various sensors to detect physiological and environmental information, for example: acceleration or movement or position sensors to detect position and/or movement; electrophysiological sensors as EMG, EOG or EEG, to detect sleep stages, REM sleep, onset of breathing cycles, and other signals; flow sensors and pressure sensors; temperature sensors; light sensors; noise and sound sensors; strain gauges, for example to detect strains of the hoses or headgear straps.

With various features have been described in the context of the individual embodiments, it should be appreciated that such various features described in separate embodiments are amenable to combination with one another. As such, it should be appreciated that the various features described herein above may be combined with one another.

A number of implementations have been described. Nevertheless, it will be understood that various modifications may be made. Accordingly, other implementations are within the scope of the following claims.

What is claimed is:

1. A breathing apparatus comprising:
an air delivery assembly;
at least one air supply tube, operatively connected to the air delivery assembly, the air supply tube including at least one outside portion being substantially flat relative to other outside portions of the air supply tube; and
a nasal interface comprising a resilient pad, operatively connected to the air delivery assembly, having first and second air passages formed therein and including a surface configured to sealingly engage respective ones of the first and second air passages.

2. The breathing apparatus according to claim 1, further comprising a plurality of nasal prongs in fluid connection with respective ones of the air passages.

3. The breathing apparatus according to claim 1, wherein the at least one air supply tube has a generally D shape.

4. The breathing apparatus according to claim 3, wherein the at least one air supply tube includes a plurality of lumens.

5. The breathing apparatus according to claim 2, wherein the nasal prongs are configured to engage an inner portion of a user's nose.

6. The breathing apparatus according to claim 5, wherein the nasal prongs are configured to fit within respective ones of the air passages.

7. The breathing apparatus according to claim 1, wherein the at least one air supply tube comprises a pre-shaped hose configured to aid holding the first and second air passages in sealing engagement with a user's nose.

8. The breathing apparatus according to claim 7, comprising two air supply tubes operatively connected to the air delivery assembly and configured to engage opposing portions of a user's head.

9. The breathing apparatus according to claim 1, further including an adhesive formed on the surface.

10. The breathing apparatus according to claim 1, further comprising a plurality of adhesive strips configured to attach to sides of a user's nose.

11. The breathing apparatus according to claim 1, wherein the resilient pad includes a gel material.

12. The breathing apparatus according to claim 1, further comprising an adhesive layer positioned to releasably connect the nasal interface and the air delivery assembly.

13. The breathing apparatus according to claim 1, wherein the air delivery assembly and the resilient pad include cooperating interlocking features for releasably coupling the nasal interface with the air delivery assembly.
